(19) United States
(12) Patent Application Publication
(10) Pub. No.: US 2024/0307002 A1
(43) Pub. Date: Sep. 19, 2024

(54) SYSTEM AND METHOD FOR TRAINING A MODEL TO MONITOR HEALTH PARAMETERS

(71) Applicant: KNOW LABS, INC., Seattle, WA (US)

(72) Inventor: JOHN CRONIN, Seattle, WA (US)

(21) Appl. No.: 18/601,494

(22) Filed: Mar. 11, 2024

Related U.S. Application Data
(60) Provisional application No. 63/490,882, filed on Mar. 17, 2023.

(51) Int. Cl. A61B 5/00; A61B 5/021; A61B 5/05

(52) U.S. Cl. CPC A61B 5/7267; A61B 5/021; A61B 5/05; A61B 5/7246

(57) ABSTRACT

A method for training a model for monitoring health parameters of a user. The method includes inputting a standard waveform database, sending radio frequency transmit signals to one or more transmitting antennas, receiving a radio frequency range signal from one or more receiving antennas, extracting a radio frequency signal waveform from the radio frequency range signal using a randomized time slice from a randomized time window, generating training data by correlating radio frequency signal waveforms to the standard waveform database, by using a randomized time slices from a randomized time windows of the radio frequency signal waveforms, using a matching technique to determine a result, and training a model using the training data, wherein the trained model correlates radio frequency signal waveforms to the standard waveform database as well as determines the correlated data to associated user's health parameter.

### FIELD

The present disclosure is generally related to systems and methods of monitoring health parameters and, more particularly, relates to training a model to monitor health parameters.

### BACKGROUND

The subject matter discussed in the background section should not be assumed to be prior art merely due to its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Diabetes is a medical condition in which a person's blood glucose level, also known as blood sugar level, is persistently elevated. Diabetes can result in severe medical complications, including cardiovascular disease, kidney disease, stroke, foot ulcers, and eye damage if left untreated. Typically, diabetes is caused by either insufficient insulin production by the pancreas, referred to as “Type 1 diabetes,” or improper insulin response by the body's cells, referred to as “Type 2 diabetes.” Further, monitoring a person's blood glucose level and administering insulin when a person's blood glucose level is too high to bring the blood glucose level down to a desired level may be part of managing diabetes. Depending on many factors, such as the severity of diabetes and the individual's medical history, a person may need to measure their blood glucose level up to ten times per day. Each year, billions of dollars are spent on equipment and supplies for monitoring blood glucose levels.

Moreover, regular glucose monitoring is a crucial component of the blood and, more specifically, diabetes care. Further, measuring blood glucose is generally invasive by giving a blood sample at a clinic or hospital. Home glucose monitoring is also possible using a variety of devices. The blood sample is obtained by pricking the skin using a tiny instrument. A glucose meter or glucometer is a tiny instrument that measures the sugar in the blood sample. The majority of glucose monitoring methods and devices require a blood sample. Currently, available glucose monitoring devices also require a blood sample, usually by pricking the skin and then using a glucose meter to determine the glucose level of a patient. These monitoring devices are not always accurate. Moreover, such monitoring devices are often prone to contamination as the patient may not be in standard conditions to give the blood sample.

Also, monitoring blood pressure, especially looking for high blood pressure (also known as hypertension), can be important to avoid a serious health problem. High blood pressure increases the risk of several health conditions, including heart disease, stroke, and kidney disease. Maintaining healthy blood pressure levels through lifestyle changes and, if necessary, medication is important. Regular check-ups with a healthcare professional are also recommended to monitor and manage blood pressure levels.

Additionally, some non-invasive devices are used to determine the health parameters of a user using radio waves of an Activated Radio Frequency range of 500 MHZ to 300 GHZ. These devices involve transmitting radio waves into the user and receiving responding frequencies to determine the health parameters. Also, some received frequencies are filtered using beamforming and the Doppler effect.

### SUMMARY

An improved system and a method to generate data for training a model to monitor health parameters with high accuracy. A method for training a model to monitor a health parameter of a user includes inputting a standard waveform database that includes a plurality of standard waveforms; sending a radio frequency transmit signal to one or more transmitting antennas; receiving a radio frequency range signal from one or more receiving antennas; extracting a radio frequency signal waveform from the radio frequency range signal using a randomized time slice from a randomized time window; generating training data by correlating the radio frequency signal waveform to a standard waveform in the standard waveform database using a matching technique to determine a result; and training a model using the training data, wherein the model correlates the radio frequency signal waveform to the standard waveform in the standard waveform database as well as determines an associated user's health parameter.

One example of a health monitoring system can include a monitoring device that includes one or more transmit antennas configured to transmit radio-frequency (RF) analyte detection signals into a user suitable for detecting an analyte in the user, and one or more receive antennas configured to detect reflected RF analyte signals that result from the RF analyte detection signals transmitted into the user. An analog-to-digital converter is connected to the one or more receive antennas and receives the reflected RF analyte signals detected by the one or more receive antennas to generate digital signals. A memory is connected to the analog-to-digital converter that stores the digital signals. The memory also stores ground truth data in the form of standard waveforms. A module is connected to the memory and is configured to: extract a randomized time slice from one of the digital signals for a randomized time window, extract a first randomized time slice from one of the standard waveforms for the randomized time window, and correlate the extracted randomized time slice from the one digital signal with the first randomized time slice from the one standard waveform using a matching technique.

An example of a health monitoring method can include generating analyte waveform signals of a user by transmitting radio-frequency (RF) analyte detection signals into the user from one or more transmit antennas for detecting an analyte and detecting, using one or more receive antennas, RF analyte waveform signals that result from the RF analyte detection signals transmitted into the user. The detected RF analyte waveform signals are converted from analog signals to digital signals using an analog-to-digital converter connected to the one or more receive antennas. A randomized time slice is extracted from one of the digital signals for a randomized time window. In addition, a first randomized time slice for the randomized time window is extracted from a standard waveform in a standard waveform database. The extracted randomized time slice from the one digital signal is then correlated with the first randomized time slice from the standard waveform using a matching technique to generate training data. If the extracted randomized time slice from the one digital signal and the first randomized time slice from the standard waveform are sufficiently correlated, a label is attached to the extracted randomized time slice from the one digital signal.

A method for training a model to monitor a health parameter of a user includes inputting a standard waveform database that includes a plurality of standard waveforms; sending a radio frequency transmit signal to one or more transmitting antennas; receiving a radio frequency range signal from one or more receiving antennas; extracting a radio frequency signal waveform from the radio frequency range signal using a randomized time slice from a randomized time window; generating training data by correlating the radio frequency signal waveform to a standard waveform in the standard waveform database using a matching technique to determine a result; training a model using the training data, wherein the model correlates the radio frequency signal waveform to the standard waveform in the standard waveform database as well as determines an associated user's health parameter.

### DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. In some examples, one element may be designed as multiple elements, or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a prior art blood pressure waveform;

FIG. 2 illustrates prior art signal processing matching techniques using convolution, cross-correlation, and autocorrelation that can be used for matching the blood pressure waveform;

FIG. 3 illustrates a block diagram of a monitoring device for monitoring a health parameter of the user, according to an embodiment;

FIGS. 4A-B illustrate a flowchart of a method performed by a device base module, according to an embodiment;

FIG. 5 illustrates a flowchart of a method performed by an input waveform module, according to an embodiment;

FIGS. 6A-C illustrate a flowchart of a method performed by a matching module, according to an embodiment;

FIG. 7 illustrates a flowchart of a method performed by a labeling module, according to an embodiment; and

FIG. 8 illustrates a flowchart of a method performed by a notification module, according to an embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred systems and methods are now described.

It is also noted that “modules” represent instructions or algorithms, etc., implemented in software or hardware.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

US 2022/0192522 is incorporated herein by reference in its entirety.

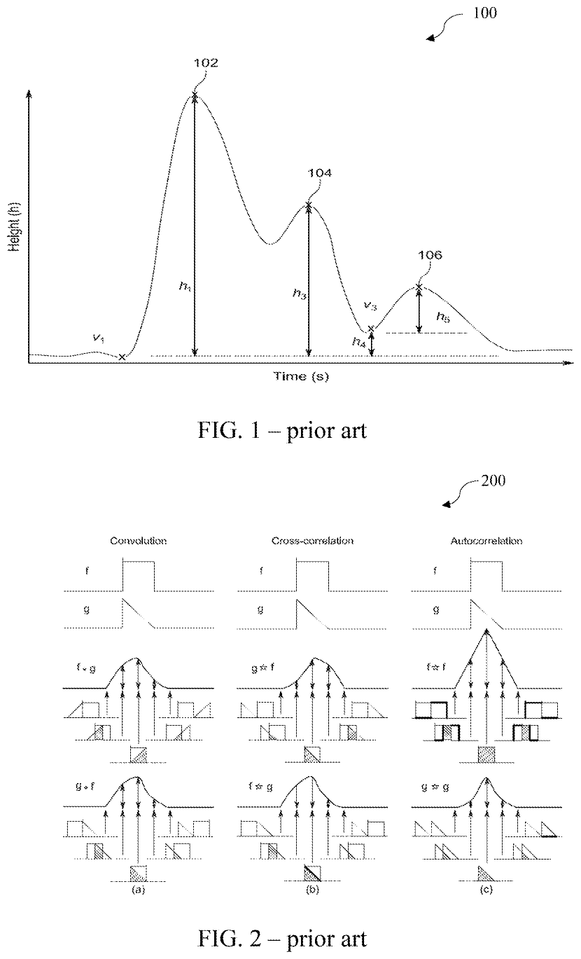

FIG. 1 illustrates a conventional blood pressure waveform 100.

The blood pressure waveform 100 corresponds to a wave of rhythmic arterial pressure. The blood pressure waveform 100 is generated when the ejection of blood takes place into an aorta of a user. The ejection of blood generates an arterial pressure wave and a blood flow wave. The arterial pressure wave is caused by the distension of elastic walls of the aorta during systole. It should be noted that blood pressure related analytes, such as sodium, potassium, aldosterone, adrenomedullin and the like, can be correlated to the arterial pressure wave. Therefore, blood pressure related analytes can be used to approximate the arterial pressure wave.

The ejection of blood from a left ventricle into the aorta of the user results in a first peak in the blood pressure waveform 100, referred to as a Percussion wave (P wave), which is shown by 102. The P wave (shown by 102) has a peak with a height of h1 for a time of t1. The time t1 is referred to as the fast ejection time of the left ventricle. It can be noted that a larger slope indicates a better performance of the heart ejection function and aorta compliance. Further, a second peak, referred to as a Tidal wave (T wave), shown by 104, appears when the blood hits the artery wall and rebounds. The T wave (shown by 104) manifests if the artery possesses excellent elasticity that reflects the low peripheral resistance of a circulatory system. The T wave (shown by 104) has a peak with a height of h3, utilized to measure the effect of the T wave (shown by 104). It may be noted that an artery with a stiff wall makes the T wave (shown by 104) propagate fast.

Accordingly, the T wave (shown by 104) merges with the P wave (shown by 102), which results in a wider P wave (shown by 106). The valley height h4 reveals a level of peripheral resistance. Peripheral resistance may be defined as the circulatory system used to create blood pressure and blood flow and is also a component of cardiac function. As the peripheral resistance increases or decreases, the valley height h4 increases or decreases. The valley height h4 and the peak h1 of the P wave 102, referred to as normalized parameter h4/h1, are employed to measure the peripheral resistance drift. Consequently, a Dicrotic wave (D wave) shown by 106 is generated when the aortic valve is closed. The D wave 106 has a peak of height h5 from the valley height of h4. The h5 is also referred to as the magnitude of the D wave 106, and the normalized parameter, h5/h1, represents the effect of the D wave 106 on the arterial system. It may be noted that h5 may decrease due to a stiff aorta or aortic regurgitation.

The shape and morphologies of the blood pressure waveform 100 may vary between users due to differences in the cardiovascular systems of an individual. Further, the morphology of the arterial pressure waveform may also differ based on the location of the user's body part where the measurement is taken. In case the location of the measurement is more distal from the heart, the P wave 102 gets steeper, and the D wave 106 is decreased. However, a mean arterial pressure (MAP) stays relatively constant irrespective of the location of the measurement. The MAP may be defined as the average blood pressure in an individual during a single cardiac cycle. Further, the morphology of the D wave 106 changes. The D wave 106 gets displaced further down the pressure curve, and rather than being a sharp interruption in the pressure descent, the D wave 106 becomes a more curved dicrotic wave. It may be noted that the change in shape from steep to curve nature of the D wave 106 is due to reflections of the arterial pressure wave rather than aortic valve closure.

One or more features may be extracted from the blood pressure waveform 100, including Heart Rate, Pulse Width, and Reflection Index. The user's heart rate may be obtained from the blood pressure waveform 100 by calculating the time interval between two consecutive P waves 102 or alternatively by calculating the time interval between two consecutive minimums. Further, the pulse width may be measured from the blood pressure waveform 100 by measuring the time interval between the points at the health height of the pulse that may be correlated with a Systematic Vascular Resistance (SVR). The SVR may be defined as the resistance against blood flow in the total vascular system.

Further, the reflection index measures pulse reflection in arteries and is related to the arterial tone, which is the magnitude of blood vessel contraction in proportion to their maximum dispensability. Also, other features may be measured from the blood pressure waveform 100, such as Large Artery Stiffness Index, Ratio of PPG Pulse Areas, Crest Time, Modified Normalized Pulse Volume (mNPV), and Heart Rate Variability.

The blood pressure waveform 100 can be created using the analytes related to blood pressure, as described above. The responded signals received from receiving antennas may be integrated with features that may be extracted via processing the blood pressure waveform 100. At least three types of features may be extracted from the blood pressure waveform 100: Timing-based features, Magnitude-based features, and Area-based features. Further, the timing-based features may provide a start time variable, end time variable, peak time variable, notch time variable, di-Peak Time variable, Peak ABP, notch ABP variable, di-Peak ABP, and height variable.

The start time variable may be defined as the start of a pulse wave. The end time variable may be defined as the end of a pulse wave. The peak time variable may be defined as the time of the P wave 102. The notch time variable may be defined as the time of the dicrotic notch. The di-Peak-Time variable may be defined as the time of the D wave 106. The peak ABP variable may be defined as the magnitude of the P wave 102. The notch ABP variable may be defined as the magnitude of the D wave 106 peaks, and the height variable may be a difference between a peak ABP and the lowest ABP.

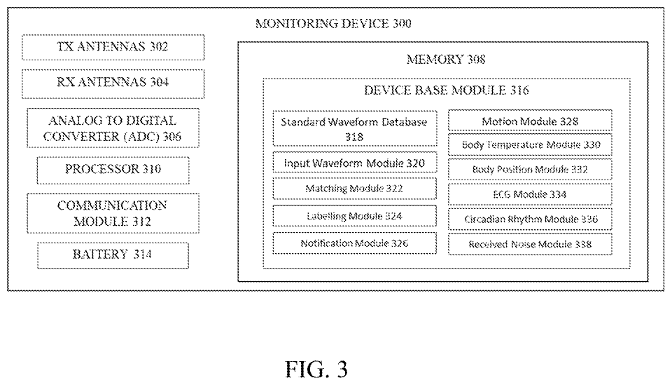

FIG. 2 illustrates examples of signal processing matching techniques 200, including convolution, cross-correlation, and autocorrelation that can be used for matching the blood pressure waveform 100 as described below.

From here on in, we will discuss blood pressure waveforms as shown in FIG. 1, which are derived from measurements of blood pressure related analytes using radio frequency signals. After the blood pressure related analytes are measured, they are transformed into blood pressure waveforms.

One or more of the signal processing matching techniques 200 can be executed to match previously existing blood pressure waveforms from a database to a current blood pressure waveform. The blood pressure measurements, that are ground truth data, and the corresponding blood pressure waveforms that may have different heights and distances between crests and troughs of the waveform may be saved in the database. The ground truth data may include the blood pressure measured from different users. For example, the ground truth data includes a first user with blood pressure 120/88 mmHg, 125/92 mmHg, and 125/90 mmHg, each having a specific type of blood pressure waveforms and thereby having different morphology. The different blood pressure waveforms are saved within the database. Further, acquired data may be taken from the user, and a blood pressure waveform may be extracted from the acquired data. The acquired data measurement may be a real-time blood pressure measurement taken from the user. Further, the blood pressure waveform from the acquired data may be matched with (i.e. compared to) the different blood pressure waveforms saved within the database to find the accurate blood pressure of the user.

The matching of the blood pressure waveform from acquired data and the different blood pressure waveforms saved within the database may be executed using any suitable signal matching technique, for example convolution, cross-correlation, or autocorrelation. The convolution technique involves the comparison of the shape of one waveform with a second waveform to generate a third waveform. The convolution technique relies on two functions, referred to as an f wave function and a g wave function, to produce a third wave function, referred to as f*g. The f wave function shows that an input signal f has a steep incline, a flat line, and a steep decline. Between the steep inclination and declination, a small kink is shown. Further, the g wave function shows that an input signal g has a steep inclination followed by a uniform declination curve. The third wave function shows that an output signal f*g has a smooth arch, which results in a slow-rising waveform being passed to the output and a slowly declining waveform.

Further, the cross-correlation technique is closely related to the convolution technique. The cross-correlation technique may be defined as a measurement technique that tracks the movements of two or more sets of time series waveforms relative to one another. The cross-correlation technique compares the multiple time series and objectively determines how well the waveforms match each other at which time period. For instance, a first input signal f has a steep incline, a flat line, and a steep decline. Between the steep inclination and declination, a small kink is shown. Further, a second input signal, g, shows a steep inclination followed by a uniform declination curve. An output signal g*f thereby results in having a smooth arch, resulting in only a slowly rising waveform being passed to the output and a slowly declining waveform.

Alternatively, in case of swapping between the first input signal and the second input signal, the morphology of the resultant waveform changes. For instance, the first input signal, g, is shown as having a steep inclination followed by a uniform declination curve, and the second input signal, f, has a steep incline, a flat line, and a steep decline. The output signal f*g thereby results in having a smooth arch which is a mirror image of the previous output signal waveform. If a variable X influences a variable Y and the two are positively correlated, then as the value of variable X increases, the value of variable Y also increases accordingly.

Further, the autocorrelation technique may be configured to represent a degree of similarity between the given input signal. The autocorrelation technique measures the relationship or changes between a variable's existing value and past value. As the degree of similarity increases between two input signals, the waveform of a resultant output signal gets sharper. For instance, an input signal f is correlated at two different time instances. The input signal f has a steep incline, a flat line, and a steep decline. Between the steep inclination and declination, a small kink is shown. As the signal waveform starts to overlap, based on the degree of similarity, the resultant waveform starts to rise. The peak of the resultant waveform thereby represents the completely overlapped state of the input signal f.

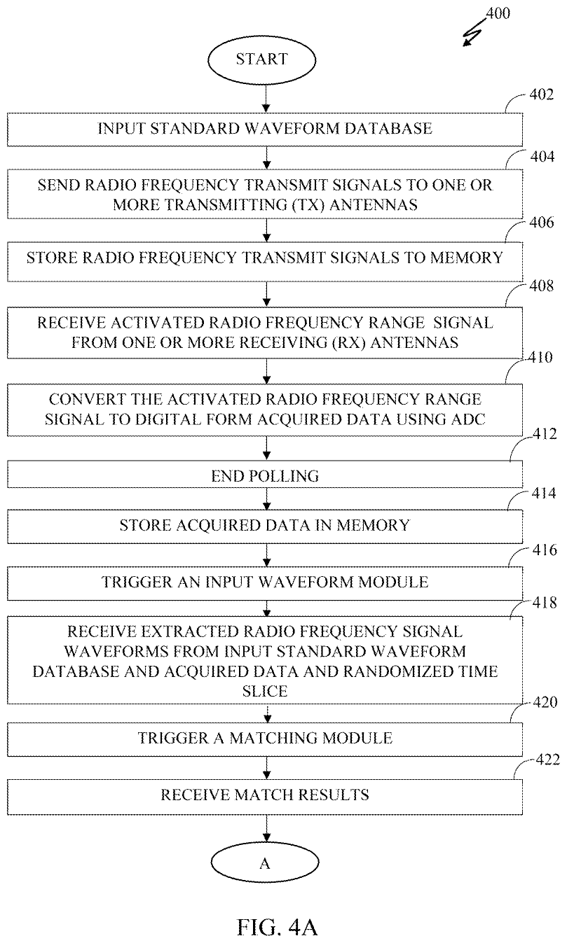

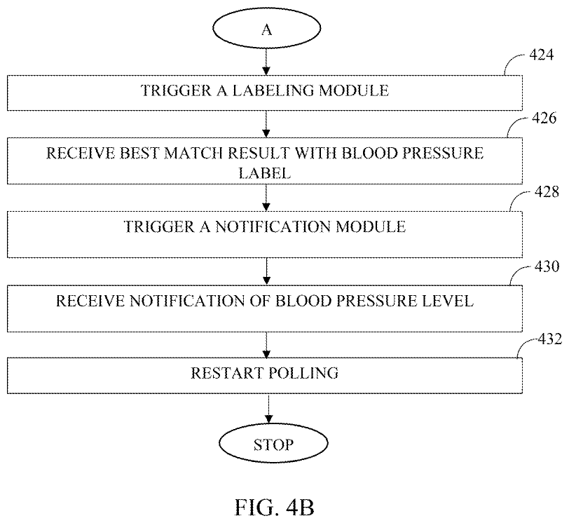

FIG. 3 illustrates a block diagram of a monitoring device 300 for use in monitoring a health parameter of the user, according to an embodiment. FIG. 3 is described in conjunction with FIGS. 4-8.

The monitoring device 300 may be worn by the user. The monitoring device 300 may be configured to collect signals in response to receiving radio frequency signals of the Activated Radio Frequency range. The monitoring device 300 may target the Activated Radio Frequency range signals to specific blood vessels, and detect resulting output signals that may correspond to the blood pressure level of the user.

In one embodiment, the monitoring device 300 may include integrated circuit (IC) devices (not shown) with transmit and/or receive antennas. The collecting of signals in response to receiving radio frequency signals of Activated Radio Frequency range from the specific blood vessels of the user involves the transmission of suitable Activated Radio Frequency range signals below the user's skin surface. Corresponding to the transmission, signals received that are a responded portion to the Activated Radio Frequency range signals are received on multiple receive antennas. Further, the monitoring device 300 may collect the signals in response to receiving the radio waves on the multiple receive antennas. The monitoring device 300 may further output a signal from the received Activated Radio Frequency range signals that correspond to the blood pressure level in the user. It can be noted that the monitoring device 300 may be worn by the user at various locations on the user's body, such as the wrist, arm, leg, etc. Further, the monitoring device 300 may process the collected signals to determine the user's health parameters.

In one embodiment, the monitoring device 300 for monitoring the blood pressure level of the user using the Activated Radio Frequency range signals involves transmitting Activated Radio Frequency range signals below the skin surface, receiving a responded portion of the Activated Radio Frequency range signals on multiple receive antennas, collecting a signal in response to receiving the radio waves on the multiple receive antennas, outputting a signal with filtered frequency waveform that corresponds to the blood pressure waveform and processing the collected signal to determine the blood pressure of the user.

In one embodiment, beamforming is used for modifying the radio frequency signal received from the receiving antenna. Certain frequency bands not associated with glucose or analytes related to blood pressure waveforms are eliminated in the beamforming process. In another embodiment, Doppler effect processing may be used for modifying the radio frequency signal received from the receiving antenna to determine a signal with a filtered frequency waveform. It can be noted that analog and/or digital signal processing techniques may be used to implement beamforming and/or Doppler effect processing and digital signal processing of the received signals to dynamically adjust a received beam. In another embodiment, the beamforming and the Doppler effect processing may be used together to modify the radio frequency signal received from the receiving antenna.

In one exemplary embodiment, activated Radio Frequency range signals of a higher frequency range of 122-126 gigahertz (GHz) in a shallower penetration depth are used to monitor the analytes related to blood pressure. It can be noted that the shallower penetration depth reduces undesirable responded signals such as reflections from bone and dense tissue such as tendons, ligaments, and muscle, which may reduce the signal processing burden and improve the quality of the desired signal that is generated from the location of the blood vessel. It can also be noted that bones are dielectric and semi-conductive. In addition, bones are anisotropic, so not only are bones conductive, they conduct differently depending on the direction of the flow of current through the bone. Alternatively, the bones are also piezoelectric materials. Therefore, Activated Radio Frequency range signals of a higher frequency range of 122-126 GHz in the shallower penetration depth may be used to monitor the blood pressure levels.

Further, the monitoring device 300 may comprise one or more transmission (TX) antennas 302, one or more receiving (RX) antennas 304, an analog to digital converter (ADC) 306, a memory 308, a processor 310, a communication module 312, a battery 314 and a device base module 316. In one embodiment, the monitoring device 300 may be a wearable and portable device such as, but not limited to, a cell phone, a smartwatch, a tracker, a wearable monitor, a wristband, and a personal blood monitoring device. The TX antennas 302 and the RX antennas 304 may be fabricated on a substrate (not shown) within the monitoring device 300 in a suitable configuration. In one exemplary embodiment, at least two TX antennas and at least four RX antennas are fabricated on the substrate. The TX antennas 302 and the RX antennas 304 may correspond to a circuitry arrangement (not shown) on the substrate.

Further, the ADC 306, the memory 308, the processor 310, the communication module 312, the battery 314, and the device base module 316 may be fabricated on the substrate. Further, the TX antennas 302 and the RX antennas 304 may be integrated into the circuitry arrangement. The TX antennas 302 may be configured to transmit the Activated Radio Frequency range signals at a pre-defined frequency. In one embodiment, the pre-defined frequency may correspond to a frequency range suitable for the human body. For example, the TX antennas 302 transmit Activated Radio Frequency range radio frequency signals at 122-126 GHz. Successively, the RX antennas 304 may be configured to receive a responded portion of the Activated Radio Frequency range signals.

In one embodiment, the Activated Radio Frequency range signals may be transmitted underneath the user's skin, and electromagnetic energy may be responded from many body parts such as fibrous tissue, muscle, tendons, bones, and the skin. It can be noted that effective monitoring of the blood pressure level is facilitated by an electrical response of blood molecules, such as pancreatic endocrine hormones, against the transmitted Activated Radio Frequency range signals. It will be apparent to a skilled person that the pancreatic endocrine hormones such as insulin and glucagon are responsible for maintaining blood pressure. Further, the electromagnetic energy response from the blood molecules may be received by the TX antennas 302. Further, the ADC 306 may be coupled to the RX antennas 304. The RX antennas 304 may be configured to receive the responded Activated Radio Frequency range signals. The ADC 306 may be configured to convert the Activated Radio Frequency range signal from an analog signal into a digital processor readable format.

Further, the memory 308 may be configured to store the transmitted Activated Radio Frequency range signals by the TX antennas 302 and receive a responded portion of the transmitted Activated Radio Frequency range signals from the RX antennas 304. Further, the memory 308 may also store the converted digital processor readable format by the ADC 306. In one embodiment, the memory 308 may include suitable logic, circuitry, and/or interfaces that may be configured to store a machine code and/or a computer program with at least one code section executable by the processor 310. Examples of implementation of the memory 308 may include, but are not limited to, Random Access Memory (RAM), Read Only Memory (ROM), Hard Disk Drive (HDD), and/or a Secure Digital (SD) card.

Further, the memory 308 may comprise a standard waveform database 318. The standard waveform database 318 may be configured to store the ground truth data. The ground truth data may comprise the blood pressure measurements of multiple users and the corresponding blood pressure waveforms. The blood pressure waveforms may have different heights and distances between crests and troughs, which may be saved in the standard waveform database 318. It can be noted that the ground truth data may have the blood pressure of different users. For example, Alex has a blood pressure of 120/88 mmHg, 125/92 mmHg, and 125/90 mmHg, and each of these readings have a specific type of blood pressure waveforms with different morphology. Further, the different blood pressure waveforms may be saved within the standard waveform database 318.

Further, the monitoring device 300 may comprise the device base module 316 fabricated within the memory 308. The device base module 316 may be configured to store a set of instructions for executing the computer program from the converted digital processor readable format of the ADC 306. The device base module 316 is configured to facilitate the operation of the processor 310, the memory 308, the TX antennas 302, the RX antennas 304, and the communication module 312. Further, the device base module 316 may be configured to create polling of the Activated Radio Frequency range signals.

Further, the device base module 316 may comprise an input waveform module 320, a matching module 322, a labeling module 324, and a notification module 326. The input waveform module 320 may be configured to extract one or more radio frequency (RF) signals from the ground truth data stored in the standard waveform database 318. Further, the input waveform module 320 may also be configured to extract RF signals of the acquired data of the user. In one embodiment, the input waveform module 320 may extract the waveforms of the radio frequency signals from the ground truth data and the acquired data of the user from a randomized time slice.

Further, the input waveform module 320 may store the extracted one or more RF signals from the ground truth data and the extracted RF signals of the acquired data of the user from the randomized time slice in the memory 308. For example, input waveform module 320 saves the RF signal of 122 GHz between time slices of 4-6 nanoseconds (ns) from the ground truth data and saves the RF signal of 124 GHz between time slices of 4-6 nanoseconds (ns) of the acquired data in the memory 308. In one embodiment, ground truth data may be frequent blood pressure measurements from a standard blood pressure measuring device.

Further, the matching module 322 may be configured to match the RF signals saved in the memory 308 with the ground truth data and the acquired data using a suitable matching technique, such as the convolution technique. In one embodiment, the matching module 322 may execute the convolution technique by matching the waveform of the RF signals. Further, the result extracted from the convolution technique may be saved in the memory 308. For example, a waveform of the RF signal from the ground truth data X has three loops, with a first loop defining a P wave having a distance from the base of the waveform as h1, and a second loop as a T wave has a distance from the base as h3 and a third loop D wave has a distance from the base as h2. In one embodiment, the P wave and the D wave are constantly visible in blood pressure waveforms across multiple users, and the T may not be visible in some cases. It may be noted that the P wave and D wave may be used in further examples. Further, another waveform of the RF signal Y of acquired data between time slice 4-6 ns has a first loop defined as a P wave having distance from the base of the waveform as h₁₊₁ and a third loop as a D wave has a distance from the base as h₂₊₁. The convolution technique generates an output signal X*Y which results in having the P wave with a distance from the base of the waveform as between h₁ and h₁₊₁, and the D wave has a distance from the base as between h₂ and h₂₊₁.

In one exemplary embodiment, the standard waveform database 318 is stored with more waveforms of the RF signals from ground truth data between the time slice of 4-6 ns, the matching module 322 initializes to compare the waveform of an increment RF signal of the ground truth data with the waveform of the RF signal of the acquired data from the user. In another exemplary embodiment, the matching module 322 is configured to automatically repeat the process of the convolution technique of matching until the time slice of 4-6 ns is complete. The matching module 322 may also be configured in one embodiment to match the waveform from the ground truth data and the acquired data using the cross-correlation technique and/or another matching technique.

For example, a waveform of the RF signal from the ground truth data has three loops, with a first loop defining a P wave 102 of FIG. 1 having distance from the base of the waveform as h1, and a second loop as a T wave 104 of FIG. 1 has a distance from the base as h3 and a third loop D wave 106 of FIG. 1 has a distance from the base as h2. Further, another waveform of the RF signal Y of acquired data between time slice 4-6 ns has a first loop defined as a P wave having distance from the base of the waveform as h₁₊₁ and a second loop as a D wave has a distance from the base as h₂₊₁. The cross-correlation technique generates an output signal X*Y which results in having the P wave with a distance from the base of the waveform as between h₁ and h₁₊₁, and the D wave has a distance from the base as between h₂ and h₂₊₁. Successively, the matching module 322 may be configured to match each waveform of the ground truth data and the acquired data in the time slice 4-6 ns and determine a perfect or close match. The close matches of the waveforms may be known as the result of the cross-correlation technique and may be saved in the memory 308.

In another embodiment, the matching module 322 may also be configured to match the waveforms using the autocorrelation technique. For example, a waveform of an input signal X from the ground truth data has a steep incline, a flat line, and a steep decline. As the input signal's waveform starts to overlap, based on the degree of similarity, the resultant waveform starts to rise. The peak of the resultant waveform thereby represents the completely overlapped state of the input signal X.

Further, the labeling module 324 may be configured to label each of the closely matched waveforms of the RF signals from the ground truth data and the acquired data. For example, the resultant waveform X*Y is the best match between the waveforms of the ground truth data and the acquired data between the time slice 4-6 ns. The resultant waveform has a specified morphology that is, the resultant waveform has a height of the P wave as h1, the height of the T wave as h2, and the height of the D wave as h3. The resultant waveform corresponds to the blood pressure measurement of 122/88 mmHg. Further, the notification module 326 may be configured to send notification of the determined label of the closely matched waveform. Further, the label may be extracted to determine the user's blood pressure.

In an alternative embodiment, the RF signals from the ground truth data and the acquired data may also be matched using mathematical models. The mathematical models comprise artificial intelligence protocols such as artificial intelligence (AI) algorithms, that may be configured to extract morphological data for each radio frequency waveform. In one embodiment, the RF signal waveforms may also be modeled as a trigonometric polynomial. The mathematical model may be configured to identify or calculate the trigonometric polynomial for each RF signal waveform. In an embodiment, the RF signal waveforms may also be modeled as the trigonometric polynomial using Fourier analysis. In an embodiment, multiple features may be extracted from the identified trigonometric polynomial for determining health parameter values.

In some embodiments, the device base module 316 may utilize a motion module 328 that includes at least one sensor from the group of an accelerometer, a gyroscope, an inertial movement sensor, or other similar sensor. The motion module 328 may have its own processor or utilize the processor 310 to calculate the user's movement. Motion from the user will change the blood volume in a given portion of their body and the blood flow rate in their circulatory system. This may cause noise, artifacts, or other errors in the real-time signals received by the RX antennas 304. The motion module 328 may compare the calculated motion to a motion threshold stored in memory 308. For example, the motion threshold could be movement of more than two centimeters in one second. The motion threshold could be near zero to ensure the user is stationary when measuring to ensure the least noise in the RF signal data. When calculated motion levels exceed the motion threshold, the motion module 328 may flag the RF signals collected at the time stamp corresponding to the motion as potentially inaccurate. In some embodiments, the motion module 328 may compare RF signal data to motion data over time to improve the accuracy of the motion threshold. The motion module 328 may alert the user, such as with an audible beep or warning or a text message or alert to a connected mobile device. The alert would signal the user that they are moving too much to get an accurate measurement. The motion module 328 may update the standard waveform database 318 with the calculated motion of the user that corresponds with the received RF signal data. In this manner, the motion module 328 may be simplified to just collect motion data and allow the device base module 316 to determine if the amount of motion calculated exceeds a threshold that would indicate the received RF signal data is too noisy to be relied upon for a blood glucose measurement.

The device base module 316 may utilize a body temperature module 330 that includes at least one sensor from the group of a thermometer, a platinum resistance thermometer (PRT), a thermistor, a thermocouple, or another temperature sensor. The body temperature module 330 may have its own processor or utilize the processor 310 to calculate the temperature of the user or the user's environment. The user's body temperature, the environmental temperature, and the difference between the two will change the blood volume in a given part of their body and the blood flow rate in their circulatory system. Variations in temperature from the normal body temperature or room temperature may cause noise, artifacts, or other errors in the real-time signals received by the RX antennas 304. The body temperature module 330 may compare the measured temperature to a threshold temperature stored in memory 308. For example, the environmental temperature threshold may be set at zero degrees Celsius because low temperatures can cause a temporary narrowing of blood vessels which may increase the user's blood pressure. When the measured temperature exceeds the threshold, the body temperature module 330 may flag the RF signals collected at the time stamp corresponding to the temperature as potentially being inaccurate. In some embodiments, the body temperature module 330 may compare RF signal data to temperature data over time to improve the accuracy of the temperature threshold. The body temperature module 330 may alert the user, such as with an audible beep or warning or a text message or alert to a connected mobile device. The alert would signal to the user that their body temperature, or the environmental temperature is not conducive to getting an accurate measurement. The body temperature module 330 update the standard waveform database 318 with the measured user or environmental temperature that corresponds with the received RF signal data. In this manner, the body temperature module 330 may be simplified to just collect temperature data and allow the device base module 316 to determine if the temperature measure exceeds a threshold that would indicate the received RF signal data is too noisy to be relied upon for a blood glucose measurement.

The device base module 316 may utilize a body position module 332 that includes at least one sensor from the group of an accelerometer, a gyroscope, an inertial movement sensor, or another similar sensor. The body position module 332 may have its own processor or utilize the processor 310 to estimate the user's position. The user's body position may change the blood volume in a given part of their body and the blood flow rate in their circulatory system. This may cause noise, artifacts, or other errors in the real-time signals received by the RX antennas 304. The body position module 332 may compare the estimated position to a body position threshold stored in memory 308. For example, the monitoring device 102 may be on the user's wrist, and the body position threshold may be based on the relative position of the user's hand to their heart. When a user's hand is lower than their heart, their blood pressure will increase, with this effect being more pronounced the longer the position is maintained. Conversely, the higher a user holds their arm above their heart, the lower the blood pressure in their hand. The body position threshold may include some minimum amount of time the estimated body position occurs. When the estimated position exceeds the threshold, the body position module 332 may flag the RF signals collected at the time stamp corresponding to the body position as potentially being inaccurate. In some embodiments, the body position module 332 may compare RF signal data to motion data over time to improve the accuracy of the body position threshold. The body position data may also be used to estimate variations in parameters such as blood pressure that corresponds to the body position data to improve the accuracy of the measurements taken when the user is in that position. The body position module 332 may alert the user, such as with an audible beep or warning or a text message or alert to a connected mobile device. The alert would signal to the user that their body position is not conducive to getting an accurate measurement. The body position module 332 may update the standard waveform database 318 with the estimated body position data that corresponds with the received RF signal data. In this manner, the body position module 332 may be simplified to just collect temperature data and allow the device base module 316 to determine if the body position exceeded a threshold that would indicate the received RF signal data is too noisy to be relied upon for a blood glucose measurement.

The device base module 316 may utilize an ECG module 334 that includes at least one electrocardiogram sensor. The ECG module 334 may have its own processor or utilize the processor 310 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the RX antennas 304. The ECG module 334 may compare the measured cardiac data to a threshold stored in memory 308. For example, the threshold may be a pulse above 160 bpm, as the increased blood flow volume may cause too much noise in the received RF signal data to accurately measure the blood glucose. When the ECG data exceeds the threshold, the ECG module 334 may flag the RF signals collected at the time stamp corresponding to the ECG data as potentially being inaccurate. In some embodiments, the ECG module 334 may compare RF signal data to ECG data over time to improve the accuracy of the ECG data threshold or to improve the measurement of glucose at a given point in the cycle between peak and minimum cardiac output. The ECG module 334 may alert the user, such as with an audible beep or warning or a text message or alert to a connected mobile device. The alert would signal to the user that their heart rate is not conducive to getting an accurate measurement or requires additional medical intervention. The ECG module 334 may update the standard waveform database 318 with the measured ECG data that corresponds with the received RF signal data. In this manner, the ECG module 334 may be simplified to just collect ECG data and allow the device base module 316 to determine if the ECG data exceeded a threshold that would indicate the received RF signal data is too noisy to be relied upon for a blood glucose measurement.

The device base module 316 may utilize a circadian rhythm module 336 that includes at least one sensor measuring actigraphy, wrist temperature, light exposure, and heart rate. The circadian rhythm module 336 may have its own processor or utilize the processor 310 to calculate the user's circadian health. Blood pressure follows a circadian rhythm in that it increases upon waking in the morning and decreases during sleeping at night. People with poor circadian health will often have higher blood pressure. These variations in blood pressure can cause noise, artifacts, or other errors or inaccuracies in the real-time signals received by the RX antennas 304. The circadian rhythm module 336 may compare the circadian data to a threshold stored in memory 308. For example, the threshold may be less than 6 hours of sleep in the last 24 hours. When the observed circadian health data exceeds the threshold, the circadian rhythm module 336 may flag the RF signals collected at the time stamp corresponding to circadian health as potentially being inaccurate or needing an adjustment to account for the expected increase in the user's blood pressure. In some embodiments, the circadian rhythm module 336 may compare RF signal data to sleep data over time to improve the accuracy of the circadian rhythm thresholds. The circadian rhythm module 336 may alert the user, such as with an audible beep or warning, or a text message or alert to a connected mobile device. The alert would signal to the user that their recent sleep patterns are not conducive to getting an accurate measurement. The circadian rhythm module 336 may update the standard waveform database 318 with the measured circadian data that corresponds with the received RF signal data. In this manner, the circadian rhythm module 336 may be simplified to just collect circadian rhythm data and allow the device base module 316 to determine if the measure exceeded a threshold that would indicate the received RF signal data is too noisy to be relied upon for a blood glucose measurement, or if an alternative transfer function should be used to compensate for the detected circadian health.

The device base module 316 may include a received noise module 338 that includes at least one sensor measuring background signals such as RF signals, Wi-Fi, and other electromagnetic signals that could interfere with the signals received by the RX antennas 304. The received noise module 338 may have its own processor or utilize the processor 310 to calculate the level of background noise being received. Background noise may interfere with or cause noise, artifacts, or other errors or inaccuracies in the real-time signals received by the RX antennas 304. The received noise module 338 may compare the level and type of background noise to a threshold stored in memory 308. The threshold may be in terms of field strength (volts per meter and ampere per meter) or power density (watts per square meter). For example, the threshold may be RF radiation greater than 300 μW/m2. When the background noise data exceeds the threshold, the received noise module 338 may flag the RF signals collected at the time stamp corresponding to background noise levels as potentially being inaccurate. In some embodiments, the received noise module 338 may compare RF signal data to background noise over time to improve the accuracy of the noise thresholds. The received radiation module may alert the user, such as with an audible beep or warning, a text message, or an alert to a connected mobile device. The alert would signal to the user that the current level of background noise is not conducive to getting an accurate measurement. The received noise module 338 may update the standard waveform database 318 with the background noise data that corresponds with the received RF signal data. In this manner, the received noise module 338 may be simplified to just collect background noise data and allow the device base module 316 to determine if the measure exceeded a threshold that would indicate the received RF signal data is too noisy to be relied upon for a blood glucose measurement, or if an alternative transfer function should be used to compensate for the noise.

In embodiments, one or more of memory 308, standard waveform database 318, input waveform module 320, matching module 322, the labelling module 324, the notification module 324, the motion module 328, the body temperature module 330, the body position module 332, the ECG module 334, the circadian rhythm module 336, and/or the received noise module 338 can be provided on one or more separate devices, such as a cloud server, networked device, or the like. In such embodiments, the communication module 312 can be used to communicate with the cloud server or the networked device to access the memory 308, standard waveform database 318, input waveform module 320, matching module 322, the labelling module 324, the notification module 324, the analyte adjust module 134, the motion module 328, the body temperature module 330, the body position module 332, the ECG module 334, the circadian rhythm module 336, and/or the received noise module 338 by way of any suitable network.

Further, the processor 310 may facilitate the operation of the monitoring device 300 to perform functions according to the instructions stored in the memory 308. In one embodiment, the processor 310 may include suitable logic, circuitry, interfaces, and/or code that may be configured to execute a set of instructions stored in the memory 308. The processor 310 may be configured to run the instructions obtained by the device base module 316 to perform polling. The processor 310 may be further configured to collect real-time signals transmitted from the TX antennas 302 and received by the RX antennas 304 and may store the real-time signals in the memory 308. In one embodiment, the real-time signals may be assigned as initial and updated radio frequency (RF) signals. Examples of the processor 310 may be an X86-based processor, a Reduced Instruction Set Computing (RISC) processor, an Application-Specific Integrated Circuit (ASIC) processor, a Complex Instruction Set Computing (CISC) processor, and/or other processors. The processor 310 may be a multicore microcontroller specifically designed to carry multiple operations based upon pre-defined algorithm patterns to achieve a desired result.

Further, the processor 310 may take inputs from the monitoring device 300 and retain control by sending signals to different parts of the monitoring device 300. The processor 310 may consist of a Random Access Memory (RAM) that is used to store data and other results that are created when the processor 310 is at work. It can be noted that the data is stored temporarily for further processing, such as filtering, correlation, correction, and adjustment. Moreover, the processor 310 carries out special tasks as programs that are pre-stored in the Read Only Memory (ROM). It can be noted that the special tasks carried out by the processor 310 indicate and apply certain actions which trigger specific responses.

In an alternative embodiment, the monitoring device 300 may also be communicatively coupled to a device network (not shown). The device network may be configured to receive data from the device base module 316 using the communication module 312. The data of the device base module 316 may be transmitted to a network base module (not shown). Examples of the communication module 312 may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). In one embodiment, various devices may be configured to have a communication module integrated over the circuitry arrangement to connect with the device network via various wired and wireless communication protocols, such as the cloud network. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols. In one embodiment, the battery 314 may be disposed on the substrate to power hardware modules of the monitoring device 300. The monitoring device 300 may be configured with a charging port to recharge the battery 314. It can be noted that the charging of the battery 314 may be wired or wireless means. In one embodiment, the battery 314 may include different models of lithium-ion batteries, such as CR1216, CR2016, CR2032, CR2025, CR2430, CR1220, CR1620, and CR1616.

In one embodiment, the device network may comprise a device network memory (not shown) configured to store the data received from the monitoring device 300. In one embodiment, the device network memory may be configured to store the filtered RF signal received from the RX antennas 304 of the monitoring device 300. Examples of implementation of the device network memory may include, but are not limited to, Cloud Storage, Cloud server, Random Access Memory (RAM), Read Only Memory (ROM), and/or a Secure Digital (SD) card.

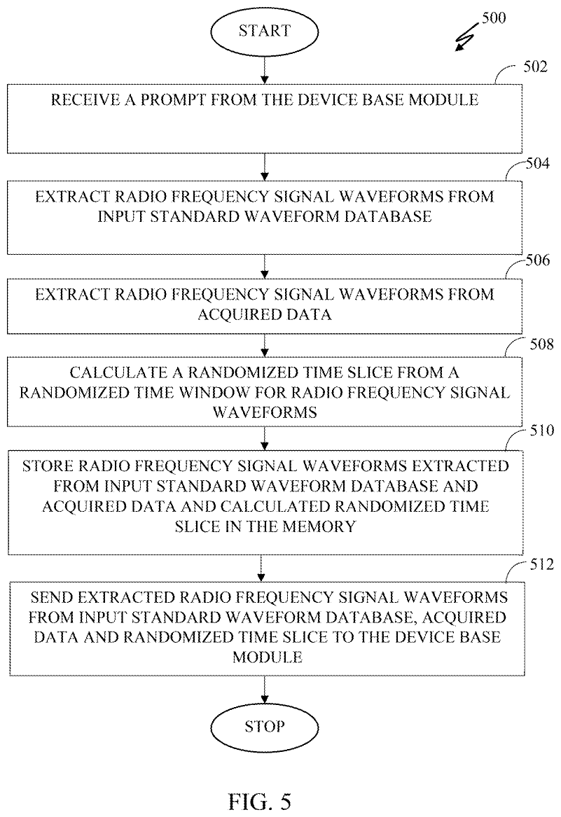

FIGS. 4A-4B illustrate a flowchart of a method 400 performed by the device base module 316, according to an embodiment. FIGS. 4A-4B are described in conjunction with FIGS. 5-8.

At first, the device base module 316 may input the standard waveform database 318 if an update is needed, at step 402. In one embodiment, there may be changes to the standard waveform database 318 with results from the training model. For example, device base module 316 inputs a standard waveform database having ground truth data as 122 GHz, 126 GHz, 130 GHz, and 140 GHz, saved in the memory 308.

In one embodiment, the ground truth data includes radio frequency signals between 122-126 GHz corresponding to individual waveforms. Each waveform has a specific type of blood pressure waveform with different morphology. These different blood pressure waveforms are saved within the standard waveform database 318.

Successively, the device base module 316 may be configured to send radio frequency transmit signals to the TX antennas 302 at step 404. For example, the device base module 316 sends a radio frequency transmitting signal of 126 GHz to the TX antennas 302. Successively, the device base module 316 may be configured to store the radio frequency transmit signals to the memory 308 at step 406. For example, the device base module 316 stores the radio frequency transmitting signal of 126 GHz to the memory 308.

Further, the device base module 316 may be configured to receive an Activated Radio Frequency range signal from the RX antennas 304 at step 408. For example, the device base module 316 receives a radio frequency signal of 123 GHz from the RX antennas 304. Successively, device base module 316 may be configured to convert the received Activated Radio Frequency range signal to form acquired data using the ADC 306 at step 410. For example, a radio signal of frequency range 123 GHz is converted into a 10-bit data signal. Successively, the device base module 316 is configured to end polling at step 412 and store the acquired data in the memory 308 at step 414. Successively, the device base module 316 may be configured to trigger the input waveform module 320 at step 416.

Further, the input waveform module 320 is described in FIG. 5. FIG. 5 illustrates a flowchart of a method 500 performed by the input waveform module 320.

At first, the input waveform module 320 may be configured to receive a prompt from the device base module 316 at step 502. Successively, the input waveform module 320 may be configured to extract radio frequency signal waveforms from the standard waveform database 318 at step 504. For example, the input waveform module 320 extracts radio frequency waveforms from the frequency signals between 122-126 GHz stored in the standard waveform database 318. The waveforms have a P wave ranging between h₁₊₁-h₁₊₃ and a D wave ranging between h₂₊₁−h₂₊₃.

Successively, the input waveform module 320 may be configured to extract radio frequency signal waveforms from the acquired data at step 506. For example, the input waveform module 320 extracts radio frequency waveforms from the radio frequency signals of 123 GHz with the P wave ranging between Q₁₊₁−Q₁₊₃ and the D wave ranging between Q₂₊₁−Q₂₊₃. Successively, the input waveform module 320 may be configured to calculate a randomized time slice from a randomized time window for radio frequency signal waveforms at step 508. For example, the input waveform module 320 calculates a randomized time slice of the 5^{th} second from the randomized time window of 30 seconds. Successively, the input waveform module 320 may be configured to store the radio frequency waveforms extracted from the standard waveform database 318 and the acquired data and the calculated randomized time slice, in the memory 308, at step 510. For example, the input waveform module 320 stores the extracted radio frequency signal waveforms from the radio frequency signals of 123 GHz having a P wave ranging between Q₁₊₁−Q₁₊₃, and a D wave ranging between Q₂₊₁−Q₂₊₃, for the randomized time slice in 5^{th} second from the randomized time window of 30 seconds.

Successively, the input waveform module 320 may be configured to send extracted radio frequency waveforms from the input standard waveform database, the acquired data, and the randomized time slice to the device base module 316 at step 512. For example, the input waveform module 320 sends extracted radio frequency signal waveforms from the radio frequency signals of 123 GHz having a P wave ranging between Q₁₊₁−Q₁₊₃, and a D wave ranging between Q₂₊₁−Q₂₊₃, for the randomized time slice in 5^{th} second from the randomized time window of 30 seconds.

Further, the device base module 316 may be configured to receive extracted radio frequency waveforms from the input standard waveform database, the acquired data, and the randomized time slice at step 418. For example, the device base module 316 receives radio frequency signal waveforms from the radio frequency signals of 123 GHz having a P wave ranging between Q₁₊₁−Q₁₊₃, and D wave ranging between Q₂₊₁−Q₂₊₃, and the randomized time slice in 5^{th} second from the randomized time window of 30 seconds. Successively, the device base module 316 may be configured to trigger the matching module 322 at step 420.

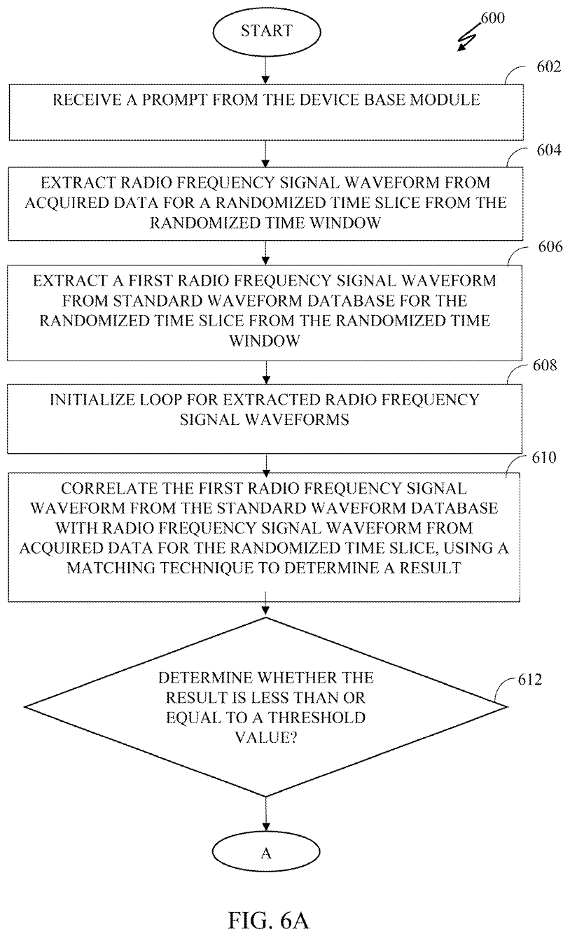

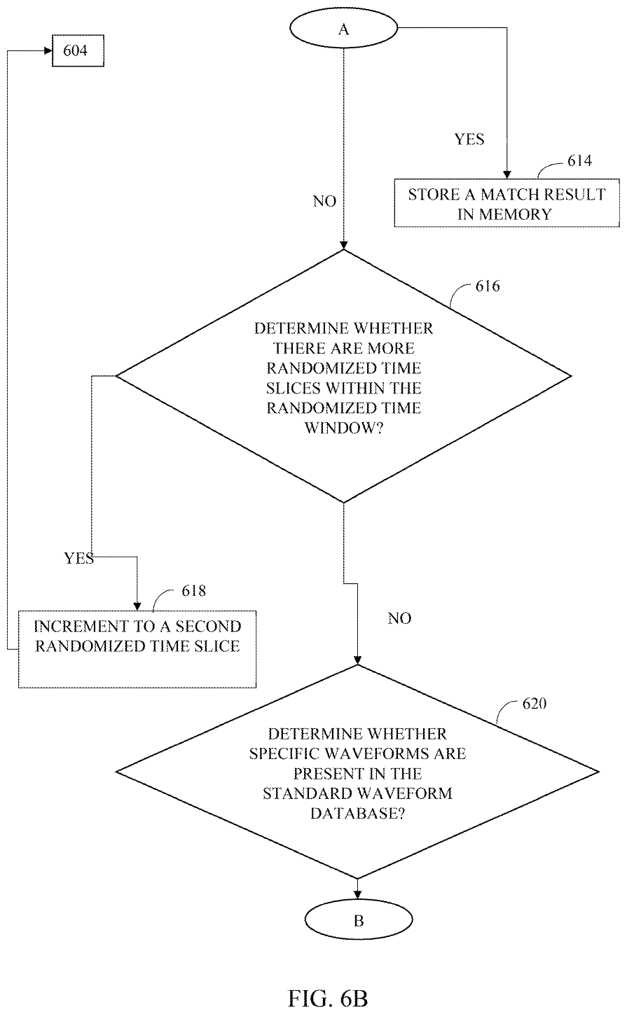

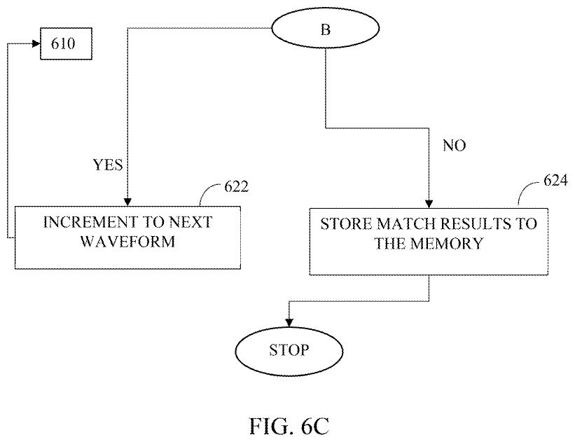

Further, the matching module 322 is described in FIGS. 6A-C. FIGS. 6A-C illustrate a flowchart of a method 600 performed by the matching module 322, according to an embodiment.

At first, the matching module 322 may be configured to receive a prompt from the device base module 316 at step 602. Successively, the matching module 322 may be configured to extract a radio frequency signal waveform from acquired data for the randomized time slice from the randomized time window at step 604. For example, the matching module 322 extracts a radio frequency signal waveform from acquired data having P waves ranging between Q₁₊₁−Q₁₊₃ and D waves ranging between Q₂₊₁−Q₂₊₃ at the time slice of the 5^{th} second. Further, the matching module 322 may be configured to extract the first radio frequency signal waveform from the standard waveform database 318 for the randomized time slice from the randomized time window at step 606. For example, the matching module 322 extracts radio frequency signal waveform from the standard waveform database 318, having a P wave ranging between h₁₊₁-h₁₊₃ and a D wave ranging between h₂₊₁−h₂₊₃ at the randomized time slice of 5^{th} second.

Successively, the matching module 322 may be configured to initialize the loop for the extracted radio frequency waveforms at step 608. For example, the matching module 322 synchronizes the timeline of the radio frequency signal waveform from the standard waveform database 318 with the radio frequency signal waveform from acquired data at exactly the 5^{th} second. Successively, the matching module 322 may correlate the first radio frequency signal waveform from the standard waveform database 318 with the radio frequency signal waveform from acquired data for the randomized time slice, using a matching technique, to determine a result at step 610. For example, the matching module 322 matches a first radio frequency signal waveform of the standard waveform database 318 having a P wave of h₁₊₁ and D wave h₂₊₁ with a radio frequency signal waveform from the acquired data having P wave Q₁₊₁ and D wave Q₂₊₁.

Successively, the matching module 322 may determine whether the result is less than or equal to a threshold value at step 612. In one embodiment, the threshold value may be equal to or less than 0.9. It can be noted that a threshold value equal to or less than 0.9 is considered a successful result. In one case, the matching module 322 determines that the result is less than or equal to the threshold value. In this case, the matching module 322 may store the result in the memory 308 at step 614. For example, the matching module 322 determines that the result is 0.7, which is less than the threshold value of 0.9 and therefore is the successful result. Further, the matching module 322 stores the successful result in the memory 308.

In another case, the matching module 322 determines that the result is more than the threshold value. In this case, the matching module 322 may proceed to step 616 to determine more randomized time slices within the randomized time window. For example, the matching module 322 determines that the result is 0.98, which is greater than the threshold value of 0.9 and therefore is unsuccessful.

Successively, the matching module 322 may determine whether there are more randomized time slices within the randomized time window at step 616. In one case, the matching module 322 determines that there are more randomized time slices within the randomized time window. In this case, the matching module 322 may increment to a second randomized time slice at step 618. For example, the matching module 322 determines that there is a 6^{th} second time slice in the randomized time window of 30 seconds. The matching module 322 increments from the first randomized time slice of the 5^{th} to the second randomized time slice of the 6^{th} second.

In another case, the matching module 322 determines that there are no more randomized time slices within the randomized time window. In this case, the matching module 322 may proceed to step 620 to determine specific waveforms present in the standard waveform database. For example, the matching module 322 determines that all randomized time slices of the randomized time window of 30 seconds have been utilized.

Successively, the matching module 322 may determine whether specific waveforms are present in the standard waveform database 318 at step 620. In one case, the matching module 322 determines that specific waveforms are present in the standard waveform database 318. In this case, the matching module 322 may proceed to increment to the next waveform at step 622. For example, the matching module 322 determines that the P wave of h₁₊₃ and the D wave h₂₊₃ waveform is present in the standard waveform database 318 at the randomized time slice of the 5^{th} second. In one embodiment, the increment to the next waveform may correspond to a second radio frequency signal waveform of the standard waveform database 318. Successively, the matching module 322 may redirect to step 622 to correlate the second radio frequency signal waveform from the standard waveform database 318 with another radio frequency signal waveform from the acquired data for the randomized time slice, using a matching technique to determine another result at step 610.

In another case, the matching module 322 determines that no specific waveforms are present in the standard waveform database 318. In this case, the matching module 322 may store the result in the memory 308 at step 624. For example, the matching module 322 determines that there are no waveforms in the standard waveform database 318 left in the 5^{th} second time slice and stores the determined result in the memory 308.

Successively, the device base module 316 may be configured to receive the determined result at step 422. For example, the device base module 316 receives that there are no waveforms in the standard waveform database 318 left in all randomized time slices of the 30-second randomized time window. Successively, the device base module 316 may be configured to trigger the labeling module 324 at step 424.

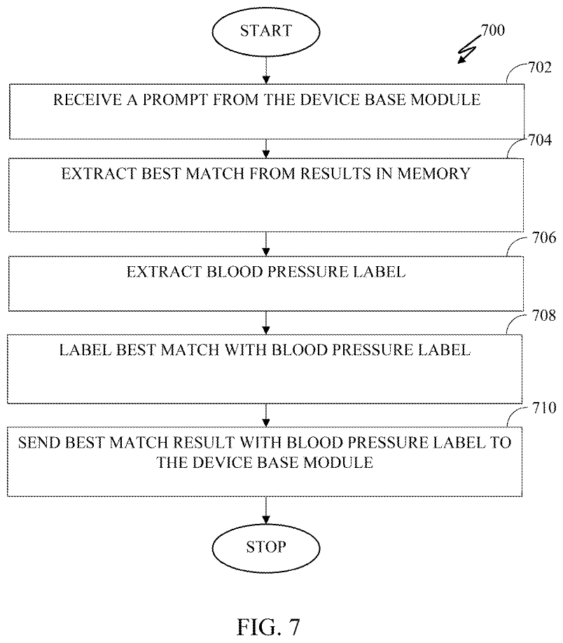

Further, labeling module 324 is described in FIG. 7. FIG. 7 illustrates a flowchart of a method 700 performed by the labeling module 324.

At first, the labeling module 324 may be configured to receive a prompt from the device base module at step 702. Successively, the labeling module 324 may be configured to extract the best match from results in memory 308 at step 704. For example, the labeling module 324 extracts the best match radio frequency signal waveforms as P wave of h₁₊₂ and D wave h₂₊₂ from the memory 308. Successively, the labeling module 324 may be configured to extract the blood pressure label at step 706. For example, the labeling module 324 extracts a blood pressure label corresponding to a blood pressure measurement of 122/85 mmHg.

Further, the labeling module 324 may be configured to label the best match with the blood pressure label at step 708. For example, the labeling module 324 labels the best match radio frequency signal waveforms as P wave of h₁₊₂ and D wave h₂₊₂ as 122/85 mmHg. Finally, the labeling module 324 may be configured to send the best match result with the blood pressure label to the device base module 316 at step 710. For example, the labeling module 324 sends the best match radio frequency waveforms as P wave of h₁₊₂ and D wave h₂₊₂ and 122/85 mmHg to the device base module 316.

Successively, the device base module 316 may be configured to receive the best match result with the blood pressure label at step 426. For example, the device base module 316 receives the best match radio frequency waveforms as P wave of h₁₊₂ and D wave h₂₊₂ and 122/85 mmHg. Successively, the device base module 316 may be configured to trigger the notification module 326 at step 428.

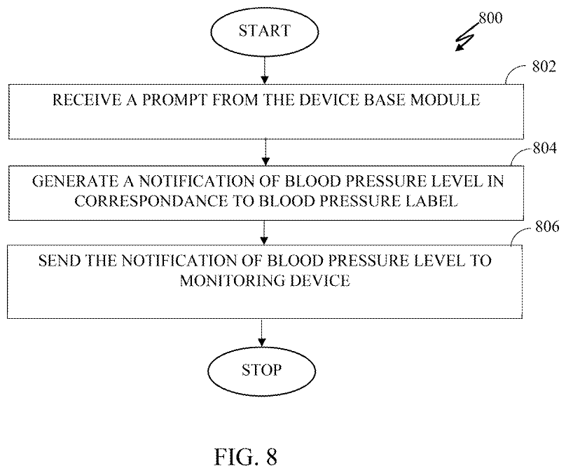

Further, the notification module 326 is described in FIG. 8. FIG. 8 illustrates a flowchart of a method 800 performed by the notification module 326, according to an embodiment.

At first, the notification module 326 may be configured to receive a prompt from the device base module 316 at step 802. Successively, the notification module 326 may be configured to generate a notification of blood pressure level in correspondence to the blood pressure label at step 804. For example, the notification module 326 generates a notification related to an actual blood pressure of Alex as 122/85 mmHg.

Successively, the notification module 326 may be configured to send a notification of blood pressure label to monitoring device 300 to alert the user at step 806. For example, the notification module 326 sends a notification to the device base module 316 related to the actual blood pressure Alex as 122/85 mmHg.

Further, the device base module 316 may be configured to receive notification of blood pressure level at step 430. For example, the device-based module 316 receives a notification related to the actual blood pressure of Alex as 122/85 mmHg over a display (not shown) of the monitoring device 300. Finally, the device base module 316 may be configured to restart polling at step 432.

It will be appreciated by those skilled in the art that changes could be made to the exemplary embodiments described above without departing from the broad inventive concept thereof. It is to be understood, therefore, that this disclosure is not limited to the particular embodiments disclosed, but is intended to cover modifications within the spirit and scope of the subject disclosure as disclosed above.

1. A health monitoring system, comprising:
a monitoring device that includes one or more transmit antennas configured to transmit radio-frequency (RF) analyte detection signals into a user suitable for detecting an analyte in the user, and one or more receive antennas configured to detect reflected RF analyte signals that result from the RF analyte detection signals transmitted into the user;
an analog-to-digital converter connected to the one or more receive antennas and receiving the reflected RF analyte signals detected by the one or more receive antennas to generate digital signals;
a memory connected to the analog-to-digital converter that stores the digital signals;
the memory stores ground truth data in the form of standard waveforms;
a module connected to the memory and configured to: extract a randomized time slice from one of the digital signals for a randomized time window, extract a first randomized time slice from one of the standard waveforms for the randomized time window, and correlate the extracted randomized time slice from the one digital signal with the first randomized time slice from the one standard waveform using a matching technique.

2. The health monitoring system of claim 1, wherein the analyte is related to blood pressure, and the standard waveforms are blood pressure waveforms.

3. The health monitoring system of claim 1, wherein the module is further configured to attach a label to the extracted randomized time slice from the one digital signal when the extracted randomized time slice from the one digital signal is correlated with the first randomized time slice from the one standard waveform.

4. The health monitoring system of claim 1, wherein the module is further configured to determine whether the correlation between the extracted randomized time slice from the one digital signal and the first randomized time slice from the one standard waveform is equal to or less than a threshold value.

5. A health monitoring method, comprising:
generating analyte waveform signals of a user by transmitting radio-frequency (RF) analyte detection signals into the user from one or more transmit antennas for detecting an analyte and detecting, using one or more receive antennas, RF analyte waveform signals that result from the RF analyte detection signals transmitted into the user;
converting the detected RF analyte waveform signals from analog signals to digital signals using an analog-to-digital converter connected to the one or more receive antennas;
extract a randomized time slice from one of the digital signals for a randomized time window;
extract a first randomized time slice for the randomized time window from a standard waveform in a standard waveform database;
correlating the extracted randomized time slice from the one digital signal with the first randomized time slice from the standard waveform using a matching technique to generate training data; and
if the extracted randomized time slice from the one digital signal and the first randomized time slice from the standard waveform are sufficiently correlated, attaching a label to the extracted randomized time slice from the one digital signal.

6. The health monitoring method of claim 5, wherein the analyte is related to blood pressure, and the standard waveform is a blood pressure waveform.

7. The health monitoring method of claim 5, comprising determining if the correlation between the extracted randomized time slice from the one digital signal and the first randomized time slice from the standard waveform is equal to or less than a threshold value.

8. The health monitoring method of claim 5, further comprising training a model using the training data.
